(19) United States
(12) Patent Application Publication
(10) Pub. No.: US 2006/0030562 A1
(43) Pub. Date: Feb. 9, 2006

(54) Use of an inhibitor of cathepsin-S-or-B to treat or prevent chronic obstructive pulmonary disease

(75) Inventors: Tao Zheng, Woodbridge, CT (US); Jack Elias, Woodbridge, CT (US); Stephen Underwood, Bedminster, NJ (US)

Correspondence Address: ROSS J. OEHLER;AVENTIS PHARMACEUTICALS INC., ROUTE 202-206, MAIL CODE: D303A, BRIDGEWATER, NJ, 08807, US

(73) Assignees: AVENTIS PHARMACEUTICALS INC., Bridgewater, NJ (US); YALE UNIVERSITY, New Haven, CT (US)

(21) Appl. No.: 11/242,411

(22) Filed: Oct. 3, 2005

Related U.S. Application Data
(63) Continuation of application No. PCT/US04/09959, filed on Apr. 1, 2004.
(60) Provisional application No. 60/459,522, filed on Apr. 1, 2003.

(51) Int. Cl. A61K 31/5377

(52) U.S. Cl. 514/234.2

(57) ABSTRACT

This invention is directed to the use of an inhibitor of cathepsin-S or -B, or composition thereof to treat or prevent chronic obstruction pulmonary disease, or physiological condition associated therewith. Such a therapy would occur using at least one of such inhibitors alone or in combination with the other, or further in combination with an anti-inflammatory agent.

### FIELD OF THE INVENTION

This invention is directed to the use of an inhibitor of cathepsin S or B to treat or prevent chronic obstruction pulmonary disease, or physiological condition associated therewith. Such a therapy would occur using at least one of such inhibitors alone or in combination with the other, or further in combination with an anti-inflammatory agent.

### BACKGROUND OF THE INVENTION

Chronic Obstructive Pulmonary Disease (COPD) is a generic term that includes several clinical syndromes including emphysema and chronic bronchitis. R. M. Senior and S. D. Shapiro, in Fisherman's Pulmonary Diseases and Disorders, R. M. Senior, Ed. (McGraw-Hill, Inc., New York, N.Y., 1998) Vol. 1, 659-681. It is a pressing clinical problem and a profound unmet medical need. In the U.S.A. it affects over 16 million people, accounts for 13% of hospitalizations and is the fourth leading cause of death. Surprisingly, the cellular and molecular events that are involved in the generation of pulmonary emphysema have only been superficially defined.

Early investigations of emphysema focused on humans with α₁-antitrypsin deficiency and intratracheal protease-based animal models. These studies demonstrated that emphysema could be caused by protease-mediated injury to the pulmonary matrix (R. M. Senior and S. D. Shapiro, in Fisherman's Pulmonary Diseases and Disorders, R. M. Senior, Ed. (McGraw-Hill Inc., New York, N.Y., 1998) Vol. 1, 659-681, R. A. Stockley, Am. J. Respir. Crit Care Med., 160, S49-S52 (1999)) and lead to the protease/antiprotease hypothesis that is still the prevailing concept in emphysema pathogenesis. This hypothesis contends that the normal lung is protected by an “antiprotease shield” and that emphysema is caused by an increase in proteases and/or a reduction in antiproteases (R. M. Senior and S. D. Shapiro, in Fisherman's Pulmonary Diseases and Disorders, R. M. Senior, Ed (McGraw-Hill, Inc., New York, N.Y., 1998) Vol. 1, 659-681, P. K. Jeffery, Am. J. Respir. Crit. Care Med., 160, 53-54 (1999)). The inflammatory response that is seen in COPD tissues is believed to be responsible for these protease/antiprotease alterations (R. M. Senior and S. D. Shapiro, in Fisherman's Pulmonary Diseases and Disorders, R. M. Senior, Ed. (McGraw-Hill, Inc., New York, N.Y., 1998) Vol. 1, 659-681, P. K. Jeffery, Am. J. Respir. Crit. Care Med., 160, 53-54 (1999)). Studies have also suggested that Type I cytokines such as gamma interferon (IFN-γ) may mediate these effects because IFN-γ producing CD8+ type I (Tc1) lymphocytes are prominent in and correlate with alveolar destruction in COPD tissues (P. K. Jeffery, Am. J. Respir. Crit. Care Med., 160, 53-54 (1999), H. A. Boushey, N. Engl. J. Med., 340, 1990-1991 (1999), M. G. Cosio and A. Guerassimov, Am. J. Respir. Crit Care Med., 160, S21-S25 (1999), T. C. O'Shaughnessy, et al., Am. J. Respir. Crit. Care Med., 155, 852-857 (1997), M. Saetta, Am. J. Respir. Crit Care Med., 160, 517-520 (1999), M. Saetta, Am. J. Respir. Crit Care Med., 160, 711-717 (1999), and M. Saetta, Am. J. Respir. Crit Care Med., 165, 1404-1409 (2002),). Also the transgenic overexpression of IFN-γ causes stimulation of cathepsin S, pulmonary emphysema with alveolar, lung enlargement and an increases in pulmonary static compliance (FIG. 2), and protease alterations in the adult murine lung (Z. Wang et al., J. Exp. Med., 192, 1587-1600 (2000)). Most recently, increased levels of structural cell apoptosis have been documented in emphysematous human tissues (J. Majo, et al. Eur. Respir. J., 17, 946-53 (May, 2001), L. Segura-Valdez, et a, Chest, 117, 684-694 (2000) and blockers of vascular endothelial cell growth factor (VEGF) have been shown to induce alveolar cell apoptosis and emphysema (Y. Kasahara, et al., J. Clin. Invest. 106, 1311-9 (December, 2000)). Surprisingly, the role of IFN-γ in the pathogenesis of Chronic Cigarette smoke-induced emphysema (CSE) and the mechanisms that mediate its emphysematous effects have not been formally defined. In addition, a common pathogenetic mechanism that links the seemingly separate protease/antiprotease, inflammatory and apoptotic theories of emphysema pathogenesis has not been formulated.

Apoptosis removes superfluous, damaged or harmful cells in a wide variety of physiologic contexts. As a result, it plays a crucial role in morphogenesis, wound healing, neoplasia, the resolution of inflammation and cellular homeostasis (G. N. Barber, Semin. Cancer Biol., 10, 103-11 (April, 2000), N. Joza, et al., Trends Genet, 18, 142-142-9 (March, 2002), and M. Leist and M Jaattela, Nat Rev. Mol. Cell. Biol., 2, 589-98 (August, 2001)). It is becoming increasingly clear, however, that dysregulation of apoptosis contributes to the pathogenesis of many human diseases and disorders (N. Joza, et al., Trends Genet., 18, 142-142-9 (March, 2002), and M. Leist and M Jaattela, Nat. Rev. Mol. Cell. Biol., 2, 589-98 (August, 2001)). This is nicely illustrated with IFN-γ, whose diverse antiviral, anti-neoplastic and immunomodulatory activities are mediated, to a significant extent, by its ability to induce lymphocyte, macrophage and neoplastic cell apoptosis (G. N. Barber, Semin. Cancer Biol., 10, 103-11, (April, 2000), E. Y. Ahn, et al., Int J. Cancer 100, 445-51, (Aug. 1, 2002), J. H. Li, et al., Am. J. Pathol. 161, 1485-95, (October, 2002), W. J. Wang, et al., J Cell Biol 159, 169-79, (Oct. 14, 2002), H. Zheng, et al., Di Yi Jun Yi Da Xue Xue Bao, 22, 1090-2, (December, 2002)). Cathepsin S is a cysteine proteinase with potent endoproteolytic activity and a broad pH profile (K. Storm van's Gravesande et al., J. Immunol., 168, 4488-94 (May 1, 2002)). It also plays an essential role in the processing of MHC II associated invariant chain in B cells and dendritic cells and has been implicated in diverse tissue remodeling responses (K. Storm van's Gravesande, et al., J. Immunol., 168, 4488-94 (May 1, 2002), G. K. Sukhova, et al, J. Clin. Invest. 102, 576-83 (Aug. 1, 1998), and S. Jormsjo, et al., Am. J. Pathol., 161, 939-45 (September, 2002)). Studies have demonstrated that a variety of cathepsins, including cathepsins B and S, are induced by IFN-γ in the murine lung (Z. Wang et al., J. Exp. Med., 192, 1587-1600 (2000)). Studies have also demonstrated that lysosomal breakdown and cathepsin B release plays an important role in TNF-mediated hepatocyte apoptosis where it induces caspase-dependent and -independent cell death pathways (S. Jormsjo, et al., Am. J. Pathol., 161, 939-45 (September, 2002), K. F. Ferri, and G. Kroemer, Nat. Cell. Biol., 3, E255-63 (November, 2001), and M. E. Guicciardi et al., J. Clin. Invest., 106, 1127-37 (November, 2000), and N. Liu, et al., Embo, J., 22, 5313-22 (Oct. 1, 2003). They are also in accord with studies that demonstrate that cathepsins can regulate p53 and cytotoxic agent-induced cellular responses (J. Lotem, and L. Sachs, Proc. Natl. Acad. Sci. USA, 93, 12507-12 (Oct. 29, 1996)), directly activate caspases (P. Schotte et al., Biocbem Biophys Res Commun 251, 379-87 (Oct. 9, 1998), K. Vancompernolle et al., FEBS Lett 438, 150-8 (Nov. 6, 1998)) and degrade subcellular matrix which would diminish the survival signals that normally come from appropriate integrin-matrix interactions (W. J. Wang, et al., J Cell Biol 159, 169-79, (Oct. 14, 2002)).

Study findings with other cells and tissues, have also demonstrate that IFN-γ is a potent stimulator of both the intrinsic and extrinsic apoptotic pathways in the lung (G. N. Barber, Semin. Cancer Biol., 10, 103-11, (April, 2000), E. Y. Ahn, et al., Int J. Cancer 100, 445-51, (Aug. 1, 2002), J. H. Li, et al., Am. J. Pathol. 161, 1485-95, (October, 2002), W. J. Wang, et al., J Cell Biol 159, 169-79, (Oct. 14, 2002), H. Zheng, et al., Di Yi Jun Yi Da Xue Xue Bao, 22, 1090-2, (December, 2002), and Y. Tesfaigzi, et al., J. Immunol., 169, 5919-25 (Nov. 15, 2002)).

In keeping with the prominent collagenolytic and elastolytic activities of cathepsin S and other cathepsins and the role of cathepsin S in tissue remodeling responses, a number of investigators have proposed that cathepsins are involved in the alveolar remodeling responses in COPD (K. Storm van's Gravesande, et al., J. Immunol., 168, 4488-94 (May 1, 2002), C. C. Taggart, et al., J. Biol. Chem., 276, 33345-52 (Sep. 7, 2001)). Surprisingly, the validity of these assumptions has not been strenuously assessed and only cathepsin L has been definitively shown to be increased in tissues from patients with emphysema (K. Takeyabu et al., Eur. Respir. J., 12, 1033-9 (November, 1998)).

The cathepsins belong to the papain superfamily of cysteine proteases. These proteases function in the normal physiological as well as pathological degradation of connective tissue. Cathepsins playa major role in intracellular protein degradation and turnover and remodeling. For example, cathepsin B, F, H, L, K, S, W, and Z have been cloned. Cathepsin K (which is also known by the abbreviation cat K) is also known as cathepsin 0 and cathepsin 02. See PCT Application WO 96/13523. Cathepsin L is implicated in normal lysosomal proteolysis as well as several disease states, including, but not limited to, metastasis of melanomas. Cathepsin S is implicated in Alzheimer's disease and certain autoimmune disorders, including, but not limited to juvenile onset diabetes, multiple sclerosis, pemphigus vulgaris, Graves' disease, myasthenia gravis, systemic lupus erythemotasus, rheumatoid arthritis and Hashimoto's thyroiditis; allergic disorders, including, but not limited to asthma; and allogenic immunbe responses, including, but not limited to, rejection of organ transplants or tissue grafts. See PCT Application WO 03/039534. Increased Cathepsin B levels and redistribution of the enzyme are found in tumors, suggesting a role in tumor invasion and matastasis. In addition, aberrant Cathepsin B activity is implicated in inflammatory airway disease and bone and joint disorders. See, D. Burnett, arch Biochem. Biophys., 317, 305-10 (1995). Cathepsin S inhibitors have also been shown to inhibit other disorders such as arteriosclerosis (G. K Sukhova, et al., J. Clin. Invest., 111, 897-906 (March, 2003)) and Th1 inflammation (N. Katunuma, et al., Biol. Chem. 384, 883-90 (June, 2003)).

In view of the aforesaid, it would be useful to have a therapy for treating or preventing COPD, and ascertaining which, if any, compounds would be useful therefor.

### SUMMARY OF THE INVENTION

This invention is directed to the use of an inhibitor of Cathepsin S or B, or composition thereof, to treat or prevent chronic obstruction pulmonary disease, or physiological condition associated therewith. Such a therapy would occur using at least one of such inhibitors alone or in combination with the other, or further in combination with an anti-inflammatory agent.

### BRIEF DESCRIPTION OF THE DRAWINGS

The above and other aspects, features, and advantages of the present invention will be better understood from the following detailed description taken in conjunction with the accompanying figures, all of which are given by way of illustration only, and are not limitative of the present invention, in which:

FIG. 1: shows CS and IFN-γ-induced apoptosis and emphysema In panels A-C, WT C57BL/6 mice were exposed to CS or room air (non-smoking) for 6 months. Their lungs were then harvested and fixed to pressure. Histologic evaluations (panel A) and morphometric evaluations of chord length (panel B) were used to quantitate alveolar size and TUNEL staining was used to quantitate apoptosis (panel C). In panels D-H, CC10-rtTA-IFN-γ transgene (+) mice generated in our laboratory and their transgene (−) littermate controls were employed. These mice were placed on normal or dox water for the noted intervals, their lungs were removed and apoptosis was evaluated. Panel (D) illustrates the percentage of total nuclei that were TUNEL (+) in transgene (−) mice on normal water (solid grey), transgene (−) mice on dox water (diagonal stripes), transgene (+) mice on normal water (horizontal stripes) and transgene (+) mice on dox water (solid black). Each value represents the mean±SEM of a minimum of 6 animals (*p<0.01 vs the other 3 groups). The TUNEL staining in lungs from transgene (−) and transgene (+) mice treated with Z-VAD-fink (Capase inhibitor, Product # G723 from Promega Corporation, 2800 Woods Hollow Road, Madison, Wis. 53711) or PBS vehicle control at 10× and 100× after 4 weeks of dox is seen in panels E and F, respectively. In panel G, total lung cells (left) and in panel H, alveolar type II cells (right) were isolated from WT and transgene (+) mice treated with dox. Apoptosis and necrosis were evaluated with annexin V and PI staining.

FIG. 2: shows effects of inhibition of apoptosis on IFN-γ-induced emphysema. In panels A, C and E, transgene (−) and transgene (+) mice were randomized to receive Z-VAD-fmk (3 μg/kg/day, via an I.P. route) or PBS vehicle control and then placed on dox for 2 weeks. In panels B, D and F we compare transgene (+) mice with wild type (+/+) and null mutant (−/−) caspase 3 loci. Lung volume (A and B), chord length (C and D) and alveolar histology (E and F) were evaluated. The values in panels A-D are the means±SEM of a minimum of 6 animals. (*p<0.01). Panels E and F are representative of 6 similar experiments (*p<0.01).

FIG. 3: shows effects of the compound of formula I (14150) disclosed in U.S. Pat. No. 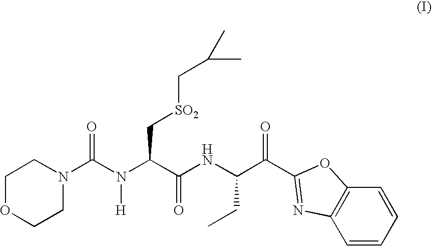
6,576,630, or a cathepsin S null mutation on IFN-γ-induced apoptosis, and emphysema In panels A, C and E transgene (−) and transgene (+) mice were randomized to receive 14150 (10 mg/kg/day twice a day by gavage) or PBS vehicle control and then placed on dox. In panels B, D and, F we compare transgene (+) mice with (+/+) and null mutant (−/−) cathepsin S loci. The percentage of cells that were TUNEL (+) (panels A and B), the chord length of the alveoli (panels C and D), and the histologic appearance of the tissues (panels E and F) were evaluated. The values in panels A-D are the mean±SEM of a minimum of 6 animals. Panels E and F are representative of 6 similar experiments (*p<0.01).

FIG. 4: shows mechanisms of apoptosis, role of cathepsin S in CSE and cathepsin S expression in smoker lungs. In panels A-C, C57B1/6 transgene (+) mice with (+/+) and (−/−) cathepsin S loci were randomized to dox water or normal water for 4 weeks. In panel A, whole lung RNA was extracted and the levels of mRNA encoding key apoptosis regulating genes were evaluated by RT-PCR In panels B and C, bioassays (top) and Western blots (bottom) are used to evaluate the activation of caspases 3 and -8 respectively. In panels D-F, wild type and cathepsin S (−/−) C57BL/6 mice were exposed to cigarette smoke (CS) or room air (non-smoking) for 6 months. Their lungs were then harvested and fixed to pressure. Histologic (panel D), morphometric (panel E) and TUNEL (panel F) evaluations were undertaken. In panels G and H, immunohistochemistry is used to compare the accumulation of cathepsin S protein in human lung tissues. In panel G, we compare the levels of cathepsin S protein in lung biopsies from populations of current smokers, former smokers and never-smokers. In panel H we compare the staining in tissues from a representative non-smoker control (left) and smoker (right). Panels A-D are representative of 3 similar experiments. The assays in panels B, C, E and F illustrate the results in a minimum of 6 mice.

FIG. 5: shows effects of selective cathepsin inhibitors. Transgene (−) and transgene (+) mice were randomized to receive apoptosis inhibitors or PBS vehicle control and placed on dox for 2 weeks. The effects of selective inhibitors of cathepsin B (CA074; (N-(L-3-trans-propylcarbamoyl-oxirane-2-carbonyl)-L-isoleucyl-L-proline), D. J. Buttle, et al., Arch. Biochem. Biophys., (December, 1992), 299(2):377-80, Peptides International Inc. Louisville, Ky. USA) and cathepsin S (14150) on apoptosis (A), lung volume (B), chord length (C) and alveolar histology (D and E) are described. The values in panels A-C are the means±SEM of a minimum of 6 animals. Panels D and E are representative of 6 similar experiments (*p<0.01).

FIG. 6: shows effect of apoptosis inhibition on IFN-γ-induced inflammation and protease activation. Transgene (−) and transgene (+) mice were randomized to receive apoptosis inhibitors or PBS vehicle control and placed on dox for 2 weeks. In panels A and B, BAL total cell, macrophage, lymphocyte and neutrophil recovery were then evaluated. In these figures we compare transgene (−) mice treated with control vehicle (horizontal stripe), transgene (−) mice that received leupeptin (diagonal stripes), transgene (+) mice that received control vehicle (solid black) and transgene (+) mice treated with Z-VAD (A) or leupeptin (B) (vertical stripe). The noted values represent the means±SEM of evaluations of a minimum of 5 animals Panels C and D illustrate the levels of mRNA encoding cathepsins (C) and MMPs (D) as assessed via RT-PCR. Real-time RTPCR quantification of the levels of mRNA encoding cathepsin B (solid bars) and cathepsin S (striped bars) are illustrated in panel E (*p<0.01, # p<0.05 vs. vehicle control treated controls).

### DETAILED DESCRIPTION OF THE INVENTION

Definitions

As used above, and throughout the description of the invention, the following terms, unless otherwise indicated, shall be understood to have the following meanings:

“COPD” as used herein is intended to include chronic bronchitis and emphysema.

“Pharmaceutically effective amount”, as used herein, means that amount of a compound or composition that will elicit the desired therapeutic effect or response or provide the desired benefit when administered in accordance with the desired treatment regimen.

“Desired therapeutic effect”, as used herein, means that the therapeutic agent or agents are continuously administered, according to the dosing schedule chosen, up to the time that the clinical or medical effect sought for the disease or condition being treated is observed by the clinician or researcher. For methods of treatment of the present invention, the pharmaceutical composition is continuously administered until the desired change in bone mass or structure is observed. In such instances, achieving an increase in bone mass or a replacement of abnormal bone structure with normal bone structure are the desired objectives. For methods of prevention of the present invention, the pharmaceutical composition is continuously administered for as long as necessary to prevent the undesired condition. Blocking the development or progression of COPD is often the objective.

“Cathepsin-S or -B inhibitor” ”, as used herein, is intended to encompass the parent compound in all its forms, i.e., optical active isomer or mixture thereof, or salt or prodrug thereof.

Non-limiting examples of cathepsin-S and -B inhibitors useful according to the invention can be found in the following patent references that are incorporated in their entirety herein: WO 2004022526; WO 2004017911; WO 2004007501; WO 2004000843; WO 2004000838; WO 2004000825; WO 2004000819; WO 2003097617; U.S. 2003203900; WO 2003086325; WO 2003075836; U.S. 2003105099; WO 2003042197; WO 2003041649; WO 2003037892; U.S. 2003073672; WO 2003029200; U.S. 2003069240; WO 2003024924; WO 2002100849; WO 2002098850; WO 2002098406; WO 2002096892; U.S. 2002137932; WO 2002032879; WO 2002020485; WO 2002020013; WO 2002020012; WO 2002020011; WO 2002014317; WO 2002014315; WO 2002014314; WO 2001096285; WO 2001089451; U.S. 2001041700; WO 2001047930; JP 2001139534; WO 2001030772; WO 2001019816; WO 2001019808; WO 2001019796; JP 2001055366; WO 2001009169; WO 2001009110; WO 2000069855; WO 2000055144; WO 2000055125; WO 2000051998; WO 2000049008; WO 2000049007; WO 2000048992; WO 9924460; JP 10036363; U.S. Pat. No. 5,691,368; WO 9621655; JP 08119983; JP 06336428; WO 9206090; EP 407017; and WO 2002040462.

In accordance with the combination therapeutic method of the present invention, a cathepsin-S or -B inhibitor can be administered at the same time in separate or combined forms, or sequentially (at different times) in any order, with an anti-inflammatory therapeutic agent The instant invention is therefore to be understood as embracing all such regimes of simultaneous or alternating administration, and the term “administering” is to be interpreted accordingly.

“Anti-inflammatory therapeutic agent” as used herein, is intended to include agents that stop or ameliorate an inflammatory condition or biological condition that has an inflammatory component assorted therewith. Thus such anti-inflammatory therapeutic agents include a short-acting beta agonist, inhaled corticosteroid, anticholinergic, long-acting beta agonist, leukotriene modifier, theophylline, or oral corticosteroid, or antibiotic that is used prophylactically to biological condition that has an inflammatory component assorted therewith.

A particular aspect of the invention provides for the cathepsin-S or -B inhibitor can be administered in the form of a pharmaceutical composition, or alone. “Pharmaceutical composition” means a composition comprising a compound of formula 1 and at least one component selected from the group comprising pharmaceutically acceptable carriers, diluents, coatings, adjuvants, excipients, or vehicles, such as preserving agents, fillers, disintegrating agents, wetting agents, emulsifying agents, emulsion stabilizing agents, suspending agents, isotonic agents, sweetening agents, flavoring agents, perfuming agents, coloring agents, antibacterial agents, antifungal agents, other therapeutic agents, lubricating agents, adsorption delaying or promoting agents, and dispensing agents, depending on the nature of the mode of administration and dosage forms. The compositions may be presented in the form of tablets, pills, granules, powders, aqueous solutions or suspensions, injectable solutions, elixirs or syrups. Exemplary suspending agents include ethoxylated isostearyl alcohols, polyoxyethylene sorbitol and sorbitan esters, microcrystalline cellulose, aluminum metahydroxide, bentonite, agar-agar and tragacanth, or mixtures of these substances. Exemplary antibacterial and antifungal agents for the prevention of the action of microorganisms include parabens, chlorobutanol, phenol, sorbic acid, and the like. Exemplary isotonic agents include sugars, sodium chloride and the like. Exemplary adsorption delaying agents to prolong absorption include aluminum monostearate and gelatin. Exemplary adsorption promoting agents to enhance absorption include dimethyl sulfoxide and related analogs. Exemplary carriers, diluents, solvents, vehicles, solubilizing agents, emulsifiers and emulsion stabilizers, include water, chloroform, sucrose, ethanol, isopropyl alcohol, ethyl carbonate, ethyl acetate, benzyl alcohol, tetrahydrofurfuryl alcohol, benzyl benzoate, polyols, propylene glycol, 1,3-butylene glycol, glycerol, polyethylene glycols, dimethylformamide, Tween® 60, Span® 60, cetostearyl alcohol, myristyl alcohol, glyceryl mono-stearate and sodium lauryl sulfate, fatty acid esters of sorbitan, vegetable oils (such as cottonseed oil, groundnut oil, corn germ oil, olive oil, castor oil and sesame oil) and injectable organic esters such as ethyl oleate, and the like, or suitable mixtures of these substances. Exemplary excipients include lactose, milk sugar, sodium citrate, calcium carbonate, dicalcium phosphate. Exemplary disintegrating agents include starch, alginic acids and certain complex silicates. Exemplary lubricants include magnesium stearate, sodium lauryl sulfate, talc, as well as high molecular weight polyethylene glycols.

The combined therapy method according to the present invention includes administrations of the therapeutics separately, simultaneously or sequentially. The choice of material in the pharmaceutical composition other than the compound of formula 1 is generally determined in accordance with the chemical properties of the active compound such as solubility, the particular mode of administration and the provisions to be observed in pharmaceutical practice. For example, excipients such as lactose, sodium citrate, calcium carbonate, dicalcium phosphate and disintegrating agents such as starch, alginic acids and certain complex silicates combined with lubricants such as magnesium stearate, sodium lauryl sulfate and talc may be used for preparing tablets.

The pharmaceutical compositions may be presented in assorted forms such as tablets, pills, granules, powders, aqueous solutions or suspensions, injectable solutions, elixirs or syrups.

“Liquid dosage form” means the dose of the active compound to be administered to the patient is in liquid form, for, example, pharmaceutically acceptable emulsions, solutions, suspensions, syrups and elixirs. In addition to the active compounds, the liquid dosage forms may contain inert diluents commonly used in the art, such solvents, solubilizing agents and emulsifiers.

Solid compositions may also be employed as fillers in soft and hard-filled gelatin capsules using such excipients as lactose or milk sugar as well as high molecular weight polyethylene glycols, and the like.

When aqueous suspensions are used they can contain emulsifying agents or agents which facilitate suspension.

The oily phase of the emulsion pharmaceutical composition may be constituted from known ingredients in a known manner. While the phase may comprise merely an emulsifier (otherwise known as an emulgent), it desirably comprises a mixture of at least one emulsifier with a fat or oil or with both a fat and oil. In a particular embodiment, a hydrophilic emulsifier is included together with a lipophilic emulsifier that acts as a stabilizer. Together, the emulsifier(s) with or without stabilizer(s) make up the emulsifying wax, and the way together with the oil and fat make up the emulsifying ointment base which forms the oily dispersed phase of the cream formulations.

If desired, the aqueous phase of the cream base may include, for example, a least 30% w/w of a polybydric alcohol, i.e. an alcohol having two or more hydroxyl groups such as propylene glycol, butane 1,3-diol, mannitol, sorbitol, glycerol and polyethylene glycol (including PEG 400) and mixtures thereof. The topical formulations may desirably include a compound that enhances absorption or penetration of the active ingredient through the skin or other affected areas.

The choice of suitable oils or fats for a formulation is based on achieving the desired properties. Thus the cream should preferably be a non-greasy, non-staining and washable product with suitable consistency to avoid leakage from tubes or other containers. Straight or branched chain, mono- or dibasic alkyl esters such as di-isopropyl myristate, decyl oleate, isopropyl palmitate, butyl stearate, 2-ethylhexyl palmitate or a blend of branched chain esters known as Crodamol CAP may be used. These may be used alone or in combination depending on the properties required. Alternatively, high melting point lipids such as white soft paraffin and/or liquid paraffin or other mineral oils can be used.

In practice, a compound/pharmaceutical compositions of the present invention may be administered in a suitable formulation to humans and animals by topical or systemic administration, including oral, inhalational rectal, nasal, buccal, sublingual, vaginal, colonic, parenteral (including subcutaneous, intramuscular, intravenous, intradermal, intrathecal and epidural), intracistemal and intraperitoneal. It will be appreciated that the preferred route may vary with for example the condition of the recipient.

“Pharmaceutically acceptable dosage forms” refers to dosage forms of the compound of the invention, and includes, for example, tablets, dragées, powders, elixirs, syrups, liquid preparations, including suspensions, sprays, inhalants tablets, lozenges, emulsions, solutions, granules, capsules and suppositories, as well as liquid preparations for injections, including liposome preparations. Techniques and formulations generally may be found in Remington's Pharmaceutical Sciences, Mack Publishing Co., Easton, Pa., latest edition.

“Formulations suitable for oral administration” may be presented as discrete units such as capsules, cachets or tablets each containing a predetermined amount of the active ingredient; as a powder or granules; as solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or molding, optionally with one or more accessory ingredients. Compressed tables may be prepared by compressing in a suitable machine the active ingredient in a free-flowing form such as a powder or granules, optionally mixed with a binder, lubricant, inert diluent, preservative, surface active or dispersing agent Molded tablets may be made by molding in a suitable machine a mixture of the powdered compounds moistened with an inert liquid diluent The tablets may optionally be coated or scored and may be formulated so as to provide slow or controlled release of the active ingredient therein.

Solid compositions for rectal administration include suppositories formulated in accordance with known methods and containing at least one compound of the invention.

If desired, and for more effective distribution, the compounds can be microencapsulated in, or attached to, a slow release or targeted delivery systems such as a biocompatible, biodegradable polymer matrices (e.g., poly(d,l-lactide co-glycolide)), liposomes, and microspheres and subcutaneously or intramuscularly injected by a technique called subcutaneous or intramuscular depot to provide continuous slow release of the compound(s) for a period of 2 weeks or longer. The compounds may be sterilized, for example, by filtration through a bacteria retaining filter, or by incorporating sterilizing agents in the form of sterile solid compositions which can be dissolved in sterile water, or some other sterile injectable medium immediately before use.

“Formulations suitable for nasal or inhalative administration” means formulations which are in a form suitable to be administered nasally or by inhalation to a patient The formulation may contain a carrier, in a powder form, having a particle size for example in the range 1 to 500 microns (including particle sizes in a range between 20 and 500 microns in increments of 5 microns such as 30 microns, 35 microns, etc.). Suitable formulations wherein the carrier is a liquid, for administrtion as for example a nasal spray or as nasal drops, include aqueous or oily solutions of the active ingredient Formulations suitable for aerosol administration may be prepared according to conventional methods and may be delivered with other therapeutic agents. Inhalative therapy is readily administered by metered dose inhalers.

“Formulations suitable for oral administration” means formulations which are in a form suitable to be administered orally to a patient. The formulations may be presented as discrete units such as capsules, cachets or tablets each containing a predetermined amount of the active ingredient; as a powder or granules; as solution or a suspension in an aqueous liquid or a non-aqueous liquid, or as an oil-in-water liquid emulsion or a water-in-oil liquid emulsion. The active ingredient may also be presented as a bolus, electuary or paste.

“Formulations suitable for parenteral administration” means formulations that are in a form suitable to be administered parenterally to a patient The formulations are sterile and include emulsions, suspensions, aqueous and non-aqueous injection solutions, which may contain suspending agents and thickening agents and anti-oxidants, buffers, bacteriostats and solutes which render the formulation isotonic, and have a suitably adjusted pH, with the blood of the intended recipient.

“Formulations suitable for rectal or vaginal administrations” means formulations that are in a form suitable to be administered rectally or vaginally to a patient Suppositories are a particular form for such formulations that can be prepared by mixing the compounds of this invention with suitable non-irritating excipients or carriers such as cocoa butter, polyethylene glycol or a suppository wax, which are solid at ordinary temperatures but liquid at body temperature and therefore, melt in the rectum or vaginal cavity and release the active component.

“Formulations suitable for systemic administration” means formulations that are in a form 20 suitable to be administered systemically to a patient. The formulation is preferably administered by injection, including transmuscular, intravenous, intraperitoneal, and subcutaneous. For injection, the compounds of the invention are formulated in liquid solutions, in particular in physiologically compatible buffers such as Hank's solution or Ringer's solution. In addition, the compounds may be formulated in solid form and redissolved or suspended immediately prior to use. Lyophilized forms are also included. Systematic administration also can be by transmucosal or transdermal means, or the compounds can be administered orally. For transmucosal or transdermal administration, penetrants appropriate to the barrier to be permeated are used in the formulation. Such penetrants are generally known in the art and include, for example, bile salts and fusidic acid derivatives for transmucosal administration. In addition, detergents may be used to facilitate permeation. Transmucosal administration may be through use of nasal sprays, for example, or suppositories. For oral administration, the compounds are formulated into conventional oral administration forms such as capsules, tablets, and tonics.

“Formulations suitable for topical administration” means formulations that are in a form suitable to be administered topically to a patient The formulation may be presented as a topical ointment, salves, powders, sprays and inhalants, gels (water or alcohol based), creams, as is generally known in the art, or incorporated into a matrix base for application in a patch, which would allow a controlled release of compound through the transdermal barrier. When formulated in an ointment, the active ingredients may be employed with either a paraffin or a water-miscible ointment base. Alternatively, the active ingredients may be formulated in a cream with an oil-in-water cream base. Formulations suitable for topical administration in the eye include eye drops wherein the active ingredient is dissolved or suspended in a suitable carrier, especially an aqueous solvent for the active ingredient Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

“Solid dosage form” means the dosage form of the compound of the invention is solid form, for example capsules, tablets, pills, powders, dragées or granules. In such solid dosage forms, the compound of the invention is admixed with at least one inert customary excipient (or carrier) such as sodium citrate or dicalcium phosphate or (a) fillers or extenders, as for example, starches, lactose, sucrose, glucose, mannitol and silicic acid, (b) binders, as for example, carboxymethylcellulose, alginates, gelatin, polyvinylpyrrolidone, sucrose and acacia, (c) humectants, as for example, glycerol, (d) disintegrating agents, as for example, agar-agar, calcium carbonate, potato or tapioca starch, alginic acid, certain complex silicates and sodium carbonate, (e) solution retarders, as for example paraffin, (f) absorption accelerators, as for example, quaternary ammonium compounds, (g) wetting agents, as for example, cetyl alcohol and glycerol monostearate, (h) adsorbents, as for example, kaolin and bentonite, (i) lubricants, as for example, talc, calcium stearate, magnesium stearate, solid polyethylene glycols, sodium lauryl sulfate, (j) opacifying agents, (k) buffering agents, and agents which release the compound(s) of the invention in a certain part of the intestinal tract in a delayed manner.

The amount of a cathepsin-S or -B inhibitor in the composition may vary widely depending upon the type of formulation, size of a unit dosage, kind of excipients and other factors known to those of skill in the art of pharmaceutical sciences. In general, a composition of a cathepsin-S or -B inhibitor for treating a COPD will comprise from 0.01% w to 110% w, preferably 0.3% w to 1% w, of active ingredient with the remainder being the excipient or excipients. Preferably the pharmaceutical composition is administered in a single unit dosage form for continuous treatment or in a single unit dosage form ad libitum when relief of symptoms is specifically required.

The concentration of the inhibitor if administered systematically is at a dose of about 1 mg to about 2000 mg for an adult of 70 kg body weight, per day. More particularly, the dose is about 10 mg to about 1000 mg/70 kg/day. Further particularly, the dose is about 100 mg to about 500 mg/70 kg/day. The concentration of the inhibitor if applied topically is about 0.1 mg to 500 mg/gm of ointment, more particularly is about 1 mg to about 100 mg/gm ointment, and further particularly is about 30 mg to about 70 mg/gm ointment. The specific concentration partially depends upon the particular inhibitor used, as some are more effective than others. The dosage concentration of the inhibitor that is actually administered is dependent at least in part upon the particular extent of progression of the COPD to be treated, the final concentration of; inhibitor that is desired at the site of action, the method of administration, the efficacy of the particular inhibitor, the longevity of the particular inhibitor, and the timing of administration relative to the severity of the disease. Preferably, the dosage form is such that it does not substantially deleteriously affect the mammal. The dosage can be determined by one of ordinary skill in the art employing such factors and using no more than routine experimentation.

The compositions and methods of the present invention are administered and carried out until the desired therapeutic effect is achieved.

Methods

Transgenic Mice

CC10-rtTA-IFN-γ mice that had been generated in our laboratory and bred for at least 10 generations onto a Balb/c background were used in these studies. These are dual transgene positive animals in which the reverse tetracycline transactivator (rtTA) drives the expression of the murine IFN-γ gene in a lung-specific and externally regulatable fashion. The transgene in these mice is activated by adding doxycycline (dox) to the animal's drinking water. These mice were maintained as dual transgene (+) heterozygotes (refered to as transgene (+) hereafter). The details of both genetic constructs, the methods of microinjection and genotype evaluation, the inducibility and the emphysematous and inflammatory phenotype of CC10-rtTA-IFN-γ mice are carried out as described in journal article entitled “Gamma Interferon Induction Of Pulmonary Emphysema In The Adult Murine Lung.” by Wang, Z. et al., J. Exp. Med., 192, 1587-1600 (2000), which is incorporated herein by reference.

Dox Water Administration.

CC 10-rtTA-IFN-γ mice and littermate controls were maintained on normal water until they were 4-6 weeks old. They were then randomized to normal water or water with dox (500 μg/ml) (dox water) as described in journal article entitled “Gamma Interferon Induction Of Pulmonary Emphysema In The Adult Murine Lung.” by Wang, Z. et al., J. Exp. Med., 192, 1587-1600 (2000), which is incorporated herein by reference.

Pharmacologic Interventions

Four to 6 week old transgene (−) and transgene (+) mice were randomized to receive the desired agent or the appropriate vehicle control. Two days later they were randomized to normal or dox water and maintained on this regimen for 2 weeks. At the end of this interval the animals were sacrificed and pulmonary phenotype assessed as described below. In experiments in which caspase-mediated apoptosis was being evaluated Z-VAD (3 μg/kg/day via an intraperitoneal (I.P.) route) was employed. In experiments in which cathepsins were being evaluated we used either the broad spectrum inhibitor Leupeptin (15 mg/kg, I.P.) (Sigma, St. Louis Mo.), the selective, irreversible and cell permeable cathepsin B inhibitor (10 mg/Kg/day LP.) or the cathepsin S inhibitor of formula I (10 mg/kg/day by gavage). In all cases comparisons to appropriate vehicle controls were undertaken.

TUNEL Evaluations

End labeling of exposed 3′-OH ends of DNA fragments in paraffin embedded tissue was undertaken with the TUNEL in situ cell death detection kit AP (Roche Diagnostics, Calif., USA) using the instructions provided by the manufacturer. After staining, 20 fields of alveoli were randomly chosen and 2000 nuclei were counted. The labeled cells were expressed as percentage of total nuclei.

Whole Lung and Type H Alveolar Epithelial Cell Isolation

Type II cells were isolated from wild type and IFN-γ transgenic mice using the methods developed by M. Corti, et al., Am. J. Respir. Cell. Mol. Biol., 14, 309-15 (1996). After anesthesia, the trachea was cannulated with 20-gauge tubing, the lungs were filled with 2 mL Dispase (Roche Diagnostic USA) followed by 0.5 mL of 1% low-melting-point agarose and the agarose was allowed to harden under crushed ice. The lungs were then placed in 2 mL of Dispase (45 min, room temperature) and transferred to Dulbecco's modified Eagle's medium (DMEM) with 25 mM HEPES with 0.01% DNAse I (Sigma, St Louis, MO). After teasing apart the digested tissue, the resulting cell suspension was sequentially filtered through 100-, 40-, and 22-um nylon mesh filters and collected after centrifugation (8 min, 130×g). Contaminating cells were removed by incubating the cell suspension in 100-mm tissue culture plates coated with a mixture of anti-CD16/CD32 and anti-CD45 monoclonal antibodies (Pharmagen USA) overnight at 4° C. and washing the non-adherent cell population. The resulting cells were>97% type II cells and were resuspended in 1× binding buffer at 1×10⁶ cells/ml for subsequent FACS analysis.

Annexin V and Propidium Iodide Apoptosis Evaluations

Type II alveolar epithelial cell poptosis was determined by annexin V and propidium iodide (PI) staining using the annexin V-FITC apoptosis detection kit (BD Biosciences, USA). Analysis was undertaken by flow cytometry (Becton Dickenson).

Bronchoalveolar Lavage (BAL) and Quantification of IFN-γ

Mice were euthanized, the trachea was isolated by blunt dissection, and tubing was secured in the airway. Two volumes of 1 mL of PBS with 0.1% BSA were then instilled and gently aspirated and pooled. Each BAL fluid sample was centrifuged, and the supernatants were stored in −70° C. until used The levels of IFN-γ were determined using a commercial ELISA (R&D Systems Inc., Minneapolis, Minn., USA) as per the manufacturer's instructions.

Histological Analysis

Animals were sacrificed via cervical dislocation, a median sternotomy was performed, and right heart perfusion was accomplished with calcium- and magnesium-free PBS to clear the pulmonary intravascular space. The lungs were then fixed to pressure (25 cm) with neutral buffered 10% formalin, fixed overnight in 10% formalin, embedded in paraffin, sectioned at 5 μm and stained. Hematoxylin and eosin (H&E) stains were performed in the Research Histology Laboratory of the Department of Pathology at Yale University School of Medicine.

Lung Volume and Compliance Assessment

Lung volume was assessed via volume displacement as described in journal article entitled “Gamma Interferon Induction Of Pulmonary Emphysema In The Adult Murine Lung.” by Wang, Z. et al., J. Exp. Med., 192, 1587-1600 (2000). In brief, the trachea was cannulated, the lungs were degassed and the lungs and heart were removed en bloc and inflated with PBS at 25 cm of pressure. The size of the lung was evaluated via volume displacement. Compliance was calculated as the change in volume divided by the change in pressure.

Morphometric Analysis

Alveolar size was estimated from the mean chord length of the airspace using techniques as described in journal article entitled “Gamma Interferon Induction Of Pulmonary Emphysema In The Adult Murine Lung.” by Wang, Z. et al., J. Exp. Med., 192, 1587-1600 (2000).

mRNA Analysis

In many experiments mRNA levels were assessed using RT-PCR as described in journal article entitled “Gamma Interferon Induction Of Pulmonary Emphysema In The Adult Murine Lung.” by Wang, Z. et al., J. Exp. Med., 192, 1587-1600 (2000). In the RT-PCR assays gene-specific primers were used to amplify selected regions of each target moiety. The amplified PCR products were detected using 1.2% agarose ethidium bromide gel electrophoresis, quantitated electronically and confirmed by nucleotide sequencing. The primers of cathepsin-B, S, H, L, MMP-2, 9, 12, 14 and T3-actin used for RT-PCR as described in journal article entitled “Gamma Interferon Induction Of Pulmonary Emphysema In The Adult Murine Lung.” by Wang, Z. et al., J. Exp. Med., 192, 1587-1600 (2000). The primers for other targeted genes are following: FAS (UP)5′-ATG CAC ACT CTG CGA TGA AG-3′, (LO)5′-TTC AGG GTC ATC CTG TCT CC-3′. FAS-L (UP)5′-CAT CAC AAC CAC TCC CAC TG-3′. (LO)5′-GTT CTG CCA GTT CCT TCT GC-3′. TRAIL (UP)5′-CTT CCG ATT TCA GGA AGC TG-3′, (LO)5′-GTT CCA GCT GCC TTT CTG TC-3′. CASPASE-3 (UP)5′-AGT CTG ACT GGA AAG CCG AA-3′, (LO)5′-AAA TTC TAG CTT GTG CGC GT-3′. CASPASE-6 (UP)5′-TTC AGA CGT TGA CTG GCT TG-3′, (LO)5′-TTT CTG TTC ACC AGC GTG AG-3′. CASPASE-8 (UP)5′-GCT GGA AGA TGA CTT GAG CC-3′, (LO)5′-CGT TCC ATA GAC GAC ACC CT-3′. CASPASE-9 (UP)5′-CCT GCT TAG AGG ACA CAG GC-3′, (LO)5′-TGG TCT GAG AAC CTC TGG CT-3′. PKC-ε (UP)5′-TAC CGG GCT ACG TTT TAT GC-3′, (LO)5′-CCA GGA GGG ACC AGT TGA TA-3′. BAK (UP)5′-CCA ACA TTG CAT GGT GCT AC-3′, (LO)5′-AGG AGT GTT GGG AAC ACA GG-3′. BAX (UP)5′-CTG CAG AGG ATG ATT GCT GA-3′, (LO)5′-GAG GAA GTC CAG TGT CCA GC-3′. BID (UP)5′-TCC ACA ACA TTG CCA GAC TA-3′, (LO)5′-CAC TCA AGC TGA ACG CAG AG-3′. BIM (UP)5′-GCC AAG CAA CCT TCT GAT GT-3′, (LO)5′-CAT TTG CAA ACA CCC TCC TT-3′. AIF (UP)5′-CAG CTG TTC CCT GAG AAA GG-3′, (LO)5′-CTC CAG CCA GTC TTC CAC TC-3′. Al-a (UP)5′-ATG GCA TCA TTA ACT GGG GA-3′, (LO)5′-TCT TCC CAA CCT CCA TTC TG-3′.

Real time quantitative RT-PCR was also employed. It was performed on The Smart Cycler II System, Cepheid, USA using Quanti-Tect SYBR Green RT-PCR Master Kit (Quagen) as per the manufacturer's instruction. This allows both reverse transcription and PCR to take place in a single reaction. The preparation of calibration curves and estimation of intrasample variation were performed as described by Yousef, G. M. et al., Cancer Res., 61, 7811-8 (2001). For each sample the amounts of the target gene and the housekeeping gene ((3-actin) were determined using calibration standard curves. The ratio of the targeted genes (cathepsin-B, -S) to p-actin was calculated as the normalized value. The following primers were used: Cathepain B (Sense: 5′-TAT CCC TAT GGA GCA TGG AG-3′, antisence 5′-GGA GTA GCC AGC TTC ACA GC-3′). Cathepsin S (Sense 5′-TGG TGG ACT GCT CAA ATG AA-3′, antisense: 5′-CCA AAG GGG AGC TGA ATG TA-3′). Reverse transcriptation (RT) was performed at 50° C. for 30 minutes and denatured at 95° C. for 5 minutes. PCR cycling conditions were initial denaturation at 95° C. for 10 minutes, followed by 32 cycles of denaturation at 95° C. for 15 seconds, annealing at 60° C. for 30 seconds and extension at 72° C. for 30 seconds.

Statistics

Normally distributed data are expressed as mean±SEM and were assessed for significance by Student's T test or ANOVA as appropriate. Data that were not normally distributed were assessed for significance using the Wilcoxon rank sum test.

The experimental establishes that an intimate relationship exists between the inflammatory, proteolytic and apoptotic events in emphysema pathogenesis. More particularly, IFN-γ's role in the pathogenesis of CSE, and the relationships between IFN-γ, protease/antiprotease alterations and apoptosis in cigarette smoke and transgenic modeling systems was established. Also demonstrated was that IFN-γ induces alveolar epithelial cell apoptosis via a cathepsin S-dependent pathway and that this apoptosis is a critical event in emphysema generation. Lastly, it was demonstrated that cathepsin S is expressed in exaggerated quantities in the lungs of cigarette smokers. These observations provide the missing link, IFN-γ induced cathepsin-dependent epithelial apoptosis, that ties the inflammation, protease/antiprotease and apoptosis theories of COPD pathogenesis together in a single pathogenic schema.

Results

Role of IFN-γ in CSE

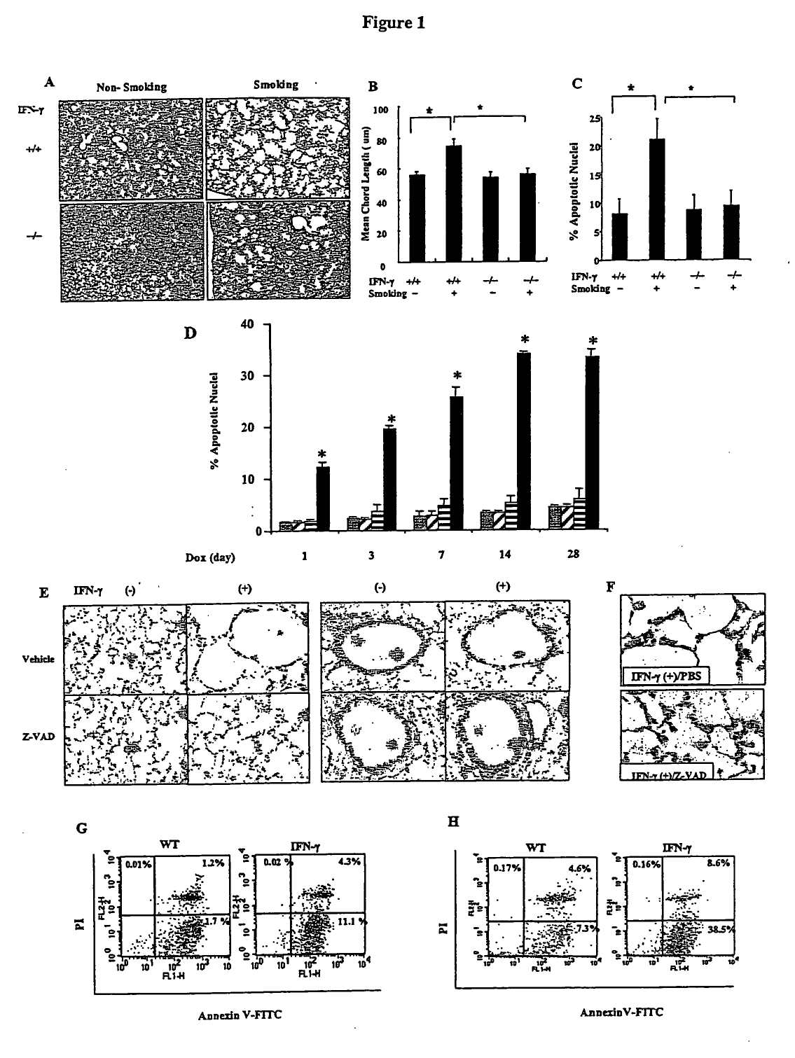

Previous studies from our laboratory demonstrated that transgenic IFN-γ induced impressive emphysema in the murine lung (Z. Wang, et al., J. Exp. Med., 192, 1587-1600 (2000)). To determine if IFN-γ played a similar critical role in CS-induced emphysema (CSE), we compared the effects of chronic CS exposure in wild type (WT) and IFN-γ null (−/−) mice. In these experiments, CS caused impressive emphysema manifest as microscopic (FIG. 1A) and morphometrically detectable alveolar enlargement (FIG. 1B). This response was associated with alveolar epithelial cell apoptosis with DNA injury detectable on TUNEL evaluation (FIG. 1C). This TUNEL staining was seen after as little as 2 months of CS exposure and persisted throughout the 6 month study interval. IFN-γ played an essential role in these responses since alveolar remodeling and epithelial cell DNA injury were both markedly ameliorated in IFN-γ (−/−) animals (FIG. 1 panels A-C).

Effect of IFN-γ on Lung Cell Apoptosis

To investigate the mechanism(s) by which IFN-γ induces these responses, TUNEL staining and FACS analysis were next used to determine if transgenic IFN-γ induced apoptosis in the murine lung. In transgene (−) mice on normal or dox water and transgene (+) mice on normal water, ≦6% of all nuclei were TUNEL stain (+) (FIG. 1D). In contrast, IFN-γ induction in transgene (+) mice caused a remarkable increase in the numbers of apoptotic nuclei (FIG. 1D). These effects were seen after as little as 1 day and peaked after 14-28 days of dox administration (FIG. 1D). At these later time points, approximately 33% of the nuclei in lung tissue sections were TUNEL positive (FIG. 1D). The majority of these cells were airway epithelial cells and type I and type II alveolar epithelial cells on light microscopic and double labeling immunohistochemical evaluations (FIG. 1 panels E and F). Similar results were obtained with FACS analysis that demonstrated increased levels of apoptosis (annexin V staining) and apoptosis with secondary necrosis (simultaneous annexin V and propidium iodide staining) in whole lung cells (FIG. 1G, left) and purified alveolar type II cells (FIG. 1H right) from transgene (+) mice treated with dox water for 2 weeks. Thus, IFN-γ is a potent inducer of epithelial apoptosis in the murine lung.

Role of Apoptosis on IFN-γ-Induced Emphysema

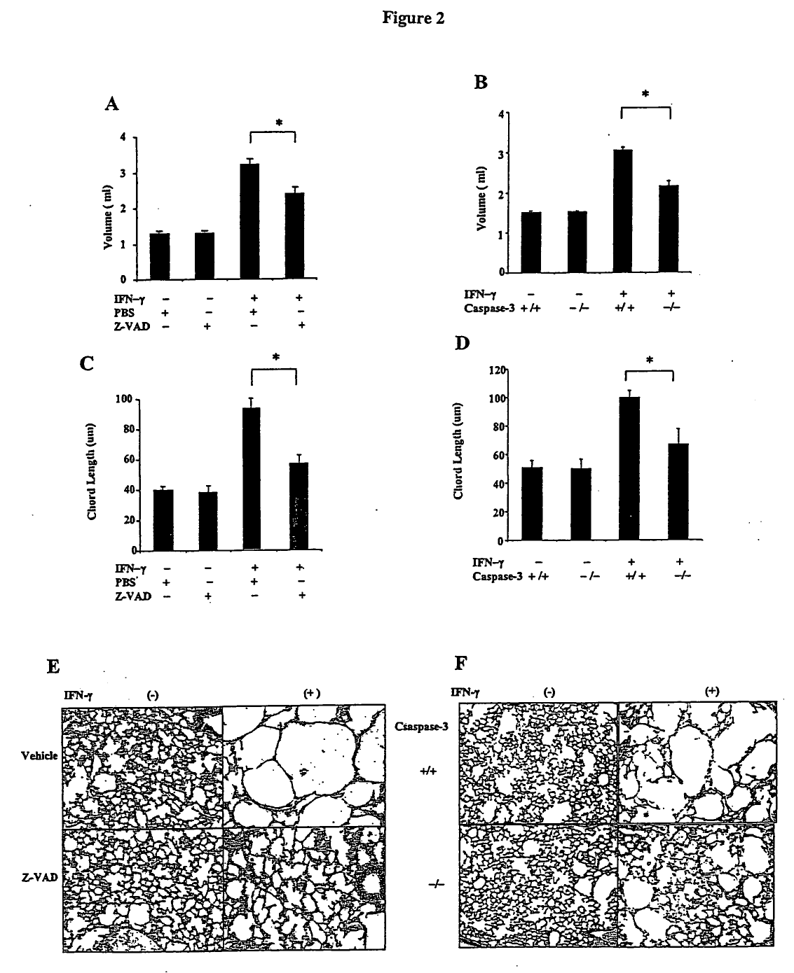

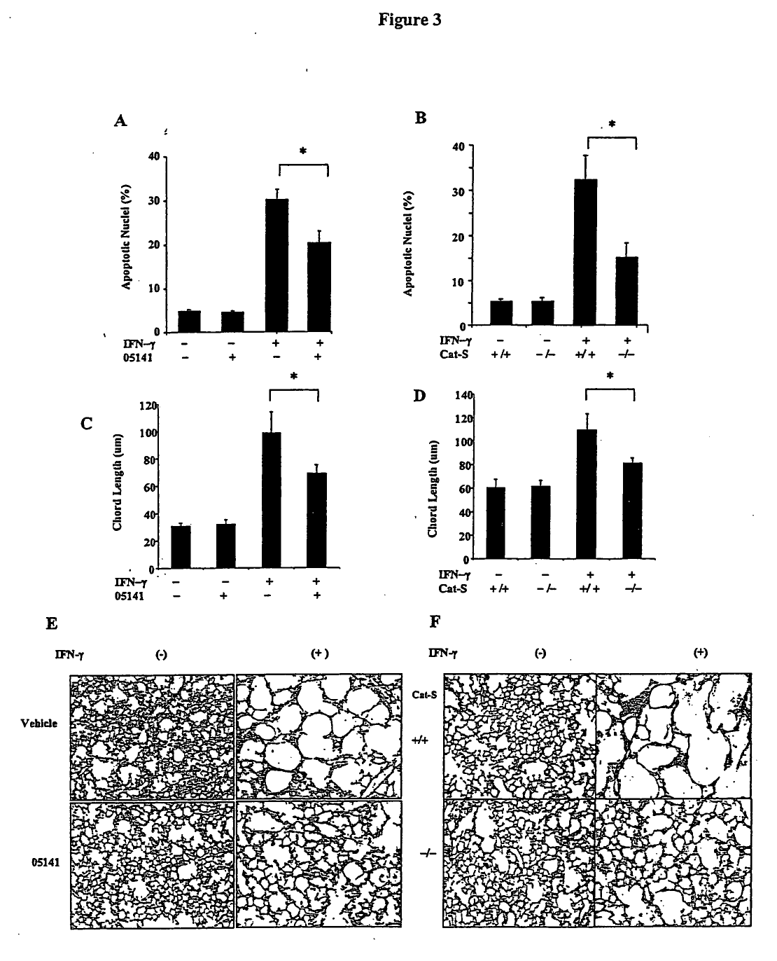

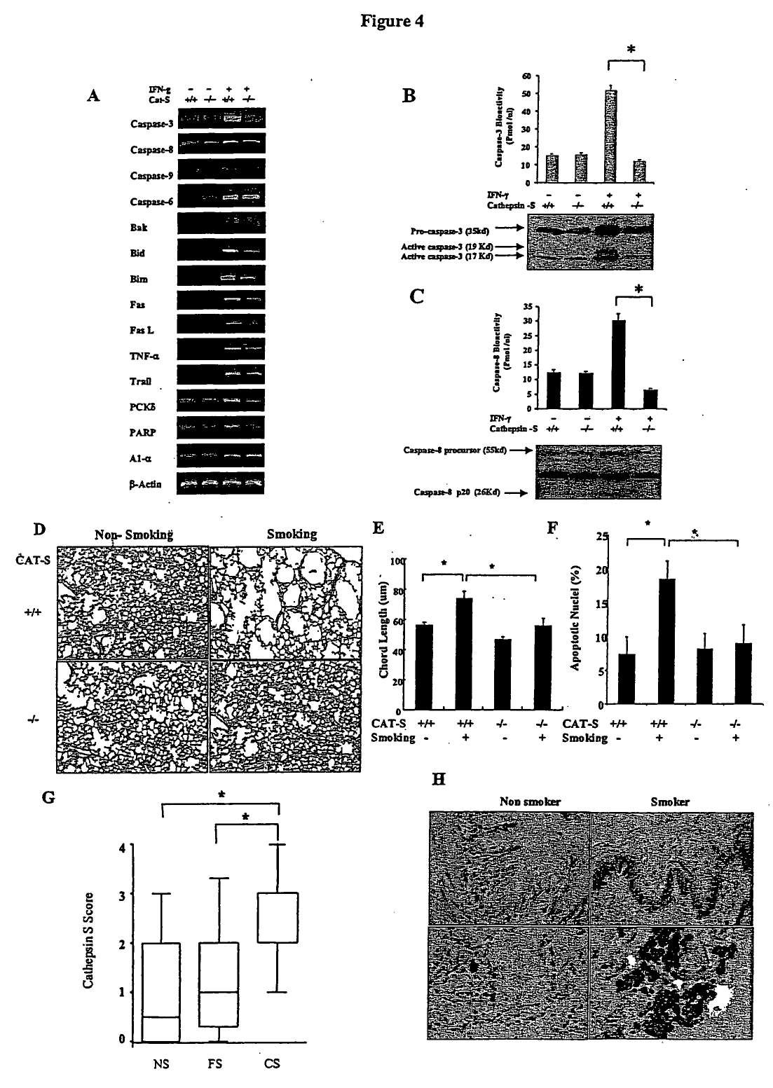

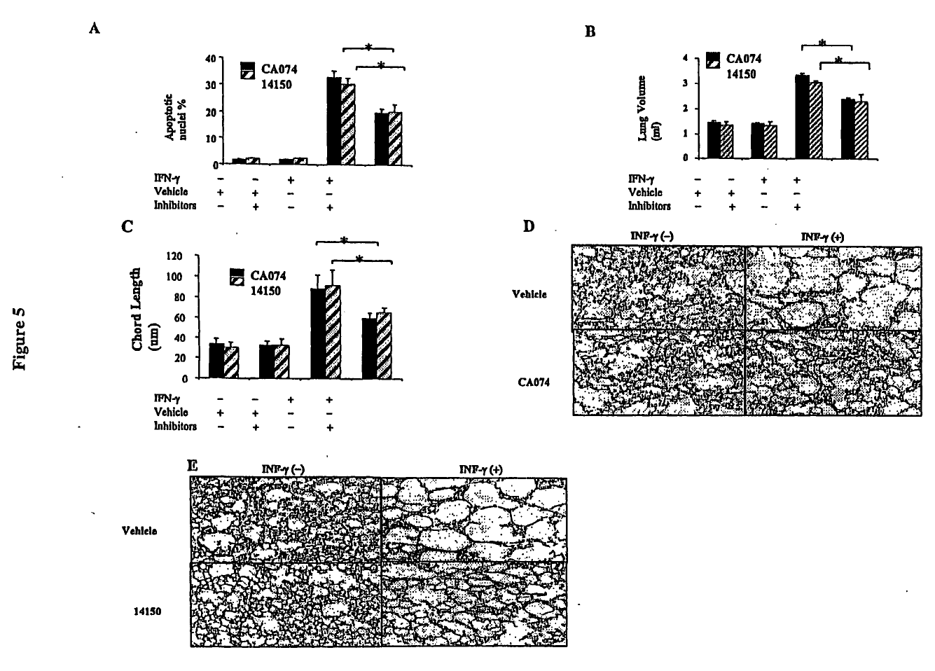

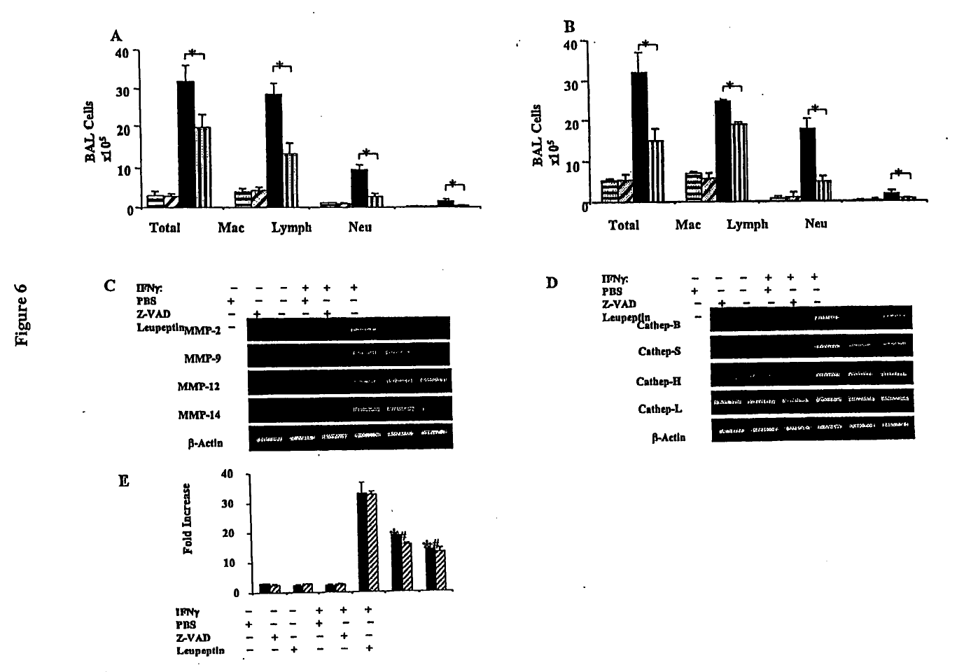

To determine if apoptosis contributed to the pathogenesis of IFN-γ-induced emphysema, we compared the emphysema generating effects of IFN-γ in mice treated with the caspase inhibitor Z-VAD-fink or vehicle control. In addition, we bred the CC10-rtTA-IFN-γ transgenic mice with caspase 3 (−/−) animals and compared the effects of IFN-γ in mice with (+/+) and (−/−) caspase 3 loci. As previously reported (Z. Wang et al., J. Exp. Med., 192, 1587-1600 (2000)), IFN-γ caused pulmonary emphysema with alveolar and lung enlargement and increases in pulmonary static compliance (FIG. 2). The chemical (Z-VAD-fmk) and the genetic (caspase 3 (−/−)) interventions decreased the levels of IFN-γ-induced apoptosis by ≧85%. They simultaneously decreased the emphysema generating effects of IFN-γ. These alterations were readily apparent in measurements of lung volume, alveolar morphometry, alveolar histology and lung compliance (FIG. 2). Thus, epithelial apoptosis is a critical event in the pathogenesis of IFN-γ-induced emphysema

Role of Cathepsin S in IFN-γ-Induced Apoptosis

We previously demonstrated that transgenic IFN-γ is a potent stimulator of cathepsin S in the murine lung (Z. Wang et al., J. Exp. Med., 192, 1587-1600 (2000)). Thus, studies were thus undertaken to determine if cathepsin S played an important role in the pathogenesis of IFN-γ-induced apoptosis and emphysema. This was done by comparing the levels of apoptosis and emphysema in IFN-γ producing transgenic mice treated with the selective cathepsin S inhibitor (14150) or its vehicle control. We also bred the IFN-γ transgenic mice with cathepsin S (−/−) mice and compared the effects of IFN-γ in mice with (+/+) and (−/−) cathepsin S loci. As noted above, IFN-γ was a potent inducer of apoptosis and emphysema in the murine lung. Importantly, treatment with 14150 or a null mutation of cathepsin S significantly inhibited these apoptosis and emphysematous responses (FIG. 3A-F). Similar decreases in apoptosis and emphysema were seen in mice treated with the broad spectrum, non-caspase, cysteine protease inhibitors leupeptin and E-64 (data shown in supplemental materials). These studies demonstrate that IFN-γ induces pulmonary epithelial cell apoptosis via a mechanism that is, at least in part, cathepsin S-dependent

Mechanism of IFN-γ-Induced Apoptosis and Regulation by Cathepsin S.

To define the mechanism of IFN-γ-induced apoptosis and the mechanism by which cathepsin S regulates this response, we compared the expression of key mediators of apoptosis in IFN-γ transgenic mice with (+/+) and (−/−) cathepsin S loci. IFN-γ-induced apoptosis was associated with significant increases in the levels of mRNA encoding key components of the extrinsic (cell death receptor) and intrinsic (mitochondrial) apoptosis pathways including Fas, Fas L, TNF α. TRAIL, Bak, Bid, Bim, caspases-3, -6, -8, -9 and protein kinase C-δ (PKCδ) (FIG. 4A). Bioassays and Western evaluations also demonstrated that IFN-γ activated caspases 3 and 8 (FIG. 4, panels B & C). Interestingly, apoptosis inducing factor (AIF) was not similarly altered (FIG. 4A). The abrogation of cathepsin S diminished the ability of IFN-γ to augment the levels of mRNA encoding Fas, FASL, TRAIL, TNF α, Bid, Bim, PKC δ and caspases-3, -8 and -9 (FIG. 4A). They also diminished the ability of IFN-γ to activate caspases 3 and 8 (FIG. 4, panels B & C). They did not, however, alter the expression of Bak, caspase 6 or A₁ (FIG. 4A). Similar alterations were induced by 14150, leupeptin and E-64. Importantly, the chemical and genetic catbepsin S manipulations and the leupeptin and E-64 treatment did not alter the levels of BAL IFN-γ, demonstrating that the decrease in apoptosis and the decrease in emphysema were not due to a decrease in transgenic cytokine production.

Role of Cathepsin S in CSE.

The studies noted above demonstrate that cathepsin S-dependent apoptosis plays a critical role in IFN-γ-induced emphysema To define the relevance of these findings to CSE we compared the responses induced by cigarette smoke in wild type and cathepsin S null mutant mice. After 6 months of CS exposure emphysema and apoptosis were readily appreciated in the control mice. Importantly, histologically and morphometrically detectable emphysema and apoptosis were markedly diminished in the cathepsin S (−/−) animals (FIG. 4, panels D-F). Thus, in accord with the findings in the transgenic system, cathepsin S also plays a critical role in the pathogenesis of CSE.

Expression of Cathepsin S in Smoker's Lungs

To evaluate the applicability of our murine findings to human COPD, we next compared the expression of cathepsin S in the lung tissue from 11 current smokers, 18 former smokers, and 5 never smokers (see Table #3 in supplemental on line information). We found that the median expression of cathepsin S was significantly different in current smokers, former smokers, and never smokers, with the highest levels of expression seen in current smokers (median scores of 2.0, 1.0, and 0.5 respectively, p=0.010) FIG. 4G). Enhanced cathepsin S staining was readily appreciated in alveolar macrophages and airway epithelial cells with lesser expression in alveolar epithelium from current smokers (FIG. 4H), whereas lower levels of cathepsin-S were seen intermittently in alveolar macrophages from non-smokers. Thus, in humans, in accord with the murine findings, cathepsin-S is expressed in an exaggerated fashion in the lungs of smokers.

The present studies were designed to further understand the importance of and the mechanism(s) by which IFN-γ generates pulmonary emphysema To accomplish this, we determined if IFN-γ plays a role in the pathogenesis of CSE and used a novel IFN-γ overexpressing transgenic mouse to define the mechanism of the emphysematous response that was noted. These studies demonstrate that IFN-γ is a critical mediator of CSE. The also demonstrate that IFN-γ is a potent inducer of epithelial apoptosis, that this apoptosis is mediated by a novel cathepsin S-dependent mechanism and that this apoptosis is a critical event in IFN-γ-induced emphysema These observations provide, for the first time, a pathogenetic construct that can unify the seemingly disparate inflammatory, protease/antiprotease and apoptotic theories of emphysema pathogenesis. By linking in a cause and effect fashion, states of inflammation, enhanced protease activity, cellular apoptosis and tissue rupture, they also define a novel pathway in tissue remodeling that may be operative in diverse biologic settings.

Apoptosis removes superfluous, damaged or harmful cells in a wide variety of physiologic contexts. As a result, it plays a crucial role in morphogenesis, wound healing, neoplasia, the resolution of inflammation and cellular homeostasis (G. N. Barber, Semin. Cancer Biol., 10, 103-11 (April, 2000), N. Joza, et al., Trends Genet., 18, 142-9 (March, 2002), M. Leist and M. Jaattela, Nat. Rev. Mol. Cell. Biol., 2,589-98 (August, 2001)). It is becoming increasingly clear, however, that dysregulation of apoptosis contributes to the pathogenesis of many human diseases and disorders (N. Joza, et al., Trends Genet., 18, 142-9 (March, 2002), M. Leist and M. Jaattela, Nat. Rev. Mol. Cell. Biol., 2, 589-98 (August, 2001)). This is nicely illustrated with IFN-γ, whose diverse antiviral, anti-neoplastic and immunomodulatory activities are mediated, to a significant extent, by its ability to induce lymphocyte, macrophage and neoplastic cell apoptosis (G. N. Barber, Semin. Cancer Biol., 10, 103-11 (April, 2000), J. H., Li, et al., Am J Pathol 161, 1485-95 (October, 2002), W. J. Wang, et al., J. Cell Biol., 159, 169-79 (Oct. 14, 2002), H. Zheng, et al., Di Yi Jun Yi Da Xue Xue Bao, 22, 1090-2 (December, 2002)). Our studies demonstrate, for the first time, that IFN-γ causes apoptosis of airway and type I and type II alveolar epithelial cells in the murine lung and that this apoptosis plays a key role in the pathogenesis of pulmonary emphysema. It is tempting to speculate from these studies that this cell death response leads to a structural weakening and cellular denudation of the alveolar septum, enhancing the ability of local proteases to digest the remaining tissue matrix and induce septal rupture. It is important to point out, however, that Z-VAD-fink administration and the null mutation of caspase 3 only partially abrogated IFN-γ-induced emphysema This suggests that apoptosis-independent events or caspase-independent apoptotic events also contribute to the pathogenesis of the IFN-γ-induced phenotype.

Cathepsin S is a cysteine proteinase with potent endoproteolytic activity and a broad pH profile ((K. Storm van's Gravesande et al., J. Immunol., 168,4488-94 (May 1, 2002))). It also plays an essential role in the processing of MHC II associated invariant chain in B cells and dendritic cells and has been implicated in diverse tissue remodeling responses (K. Storm van's Gravesande, et al., J. Immunol. 168, 4488-94 (May 1, 2002), G. K. Sukhova, et al., J. Clin. Invest, 102, 576-83 (Aug. 1, 1998, S. Jormsjo, et al., Am J. Pathol. 161, 93945 (September, 2002)). We previously demonstrated that IFN-γ-is a potent stimulator of cathepsin S in the lung (Z. Wang et al., J. Exp. Med., 192, 1587-1600 (2000)). The present studies demonstrate that the targeted null mutation or chemical inhibition of cathepsin S ameliorates IFN-γ-induced apoptosis and emphysema These are the first studies to implicate cathepsin S in apoptosis and the first to demonstrate a role for cathepsin S-mediated apoptosis in any tissue response. The demonstration that cathepsin S is involved in cellular apoptosis is in accord with studies that demonstrate that lysosomal breakdown and cathepsin B release plays an important role in TNF-mediated hepatocyte apoptosis where it induces caspase-dependent and -independent cell death pathways (L. Foghsgaard, et al., J. Cell. Biol., 153, 999-1010 (May 28, 2001), K. F. Ferri and G. Kroemer, Nat. Cell. Biol., 3, E255-63 (November, 2001), M. E. Guicciardi, et al., J. Clin. Invest., 106, 1127-37 (November, 2000), N. Liu, et al., Embo. J., 22, 5313-22 (Oct. 1, 2003)). They are also in accord with studies that demonstrate that cathepsins can regulate p53 and cytotoxic agent-induced cellular responses (J. Lotem and L. Sachs, Proc. Natl. Acad. Sci. USA, 93, 12507-12 (Oct. 29, 1996)), directly activate caspases (P. Schotte, et al., Biochem Biophys Res Commun 251, 379-87 (Oct. 9, 1998), K. Vancompernolle, et al., FEBS Lett., 438, 150-8 (Nov. 6, 1998)) and degrade subcellular matrix which would diminish the survival signals that normally come from appropriate integrin-matrix interactions (W. J. Wang, et al., J. Cell. Biol., 159, 169-79 (Oct. 14, 2002)). Additional investigation will be required to determine if the apoptotic effects of cathepsins are the result of their ability to augment intracellular apoptotic pathways, modulate extracellular matrix-cell interactions or both.

To understand the mechanism(s) by which IFN-γ induces apoptosis in the lung, we evaluated the effects of IFN-γ on the expression of key components of these pathways. These studies demonstrate that IFN-γ is a potent stimulator of Fas, Fas L, TNF γ, TRAIL, Bak, Bid, Bim Bax, PKC-δ and caspases-3, -6, -8 and -9. They also demonstrate that IFN-γ is a potent activator of caspases 3 and 8 but does not alter the levels of mRNA encoding AIF. In accord with findings with other cells and tissues, these studies demonstrate that IFN-γ is a potent stimulator of both the intrinsic and extrinsic apoptotic pathways in the lung (14, 17-20, 31). They also demonstrate that IFN-γ simultaneously induces the anti-apoptotic Al protein that may feedback to control lung epithelial cell apoptosis and/or augment the survival of the neutrophils that are recruited by IFN-γ in this disorder (32). Interestingly, when cathepsin S was genetically or chemically ablated, the ability of IFN-γ to activate cathepsins 3 and 8 and stimulate the expression of caspases 3 and 8 and Bim, Bid, Fas, TNF γ and TRAIL were markedly diminished. These studies are the first to demonstrate a role for lysosomal cathepsins, in particular cathepsin S in IFN-γ-induced apoptosis and highlight the importance of cathepsin S in IFN-γ activation of both the intrinsic and extrinsic mitochondrial apoptosis pathways.

In keeping with the prominent collagenolytic and elastolytic activities of cathepsin S and other cathepsins and the role of cathepsin S in tissue remodeling responses, a number of investigators have proposed that cathepsins are involved in the alveolar remodeling responses in COPD (K. Storm van's Gravesande, et al., J. Immunol., 168, 4488-94 (May 1, 2002), C. C. Taggart, et al., J. Biol. Chem., 276, 33345-52 (Sep. 7, 2001)). Surprisingly, the validity of these assumptions has not been strenuously assessed and only cathepsin L has been definitively shown to be increased in tissues from patients with emphysema (K. Takeyabu, et al., Eur Respir J 12, 1033-9 (November, 1998)). Our studies demonstrate, for the first time, that cathepsin S is increased in lung tissues from smokers. Importantly, they also demonstrate that IFN-γ induces emphysema, at least in part, via its ability to induce a cathepsin S-dependent tissue apoptosis response. These observations provide an attractive explanation for the disappearance of the basement membrane, matrix and cellular components of the alveolar septum during the emphysematous response. When viewed in combination, they also provide evidence that validates cathepsin S as a target against which therapies can be directed in the treatment of emphysema This is a particularly attractive prospect because cathepsin S inhibitors also inhibit other cigarette-induced associated disorders such as arteriosclerosis (G. K. Sukhova, et al., J. Clin. Invest., 111, 897-906 (March, 2003)) and Th1 inflammation (N. Katunuma, et al., Biol. Chem., 384, 883-90 (June, 2003)). Additional investigation of the therapeutic utility of apoptosis and cathepsin-based therapies for emphysema and other diseases characterized by inflammation, protease excess and tissue destruction is warranted.

Role of Cathepsin B and Cathepsin S

To begin to elucidate the contributions of specific enzymes, we compared the apoptosis and emphysema generating effects of IFN-γ in the presence and absence of CA074 and 14150 that are selective inhibitors of cathepsins B and S, respectively. As noted above, transgenic IFN-γ was a potent inducer of epithelial apoptosis and emphysema (FIG. 5). Cathepsins B and S appeared to play a significant role in this response because significant decreases in apoptosis (FIG. 5A) and emphysema (lung volumes, chord length and histology) were seen in mice treated with compounds CA074 and 14150, respectively (FIG. 5 panels, B-D). These studies demonstrate that cathepsin S B and S play critical roles in IFN-γ-induced apoptosis and emphysema.

Role of Apoptosis in IFN-γ-induced Inflammation and Proteolysis

To further understand the relationships between apoptosis and other aspects of the IFN-γ phenotype, studies were undertaken to determine if blocking apoptosis altered the ability of IFN-γ to induce inflammatory or proteolytic tissue responses. When compared to wild type mice on normal or dox water, IFN-γ production in transgene (+) mice caused a significant increase in bronchoalveolar lavage (BAL) total cell, neutrophil, lymphocyte and macrophage recovery and a patchy mononuclear tissue inflammatory response. These responses were apoptosis-dependent because Z-VAD treatment markedly ameliorated the IFN-γ-induced BAL total cell, neutrophil, lymphocyte and macrophage alterations and tissue inflammation (FIG. 6). Significant numbers of eosinophils were not noted in BAL fluids or tissues in any experimental condition. In accord with our demonstration that IFN-γ induces apoptosis via a cathepsin-dependent mechanism, similar decreases in BAL and tissue inflammation were noted when transgene (+) mice were treated with leupeptin. (FIG. 6).

When compared to wild type mice on normal or dox water, IFN-γ production in transgene (+) mice also caused a significant increase in the levels of mRNA encoding cathepsins B, S, and H and MMPs-2, 9, 12 and 14 (FIG. 6). These responses were partially apoptosis-dependent because Z-VAD treatment markedly ameliorated the IFN-γ-induced increase in the levels of mRNA encoding cathepsin B, cathepsin S and MMPs-2 and -14 (FIG. 6). The levels of mRNA encoding cathepsin H and MMPs-9 and -12 were not similarly altered (FIG. 6). Interestingly, leupeptin also decreased the ability of IFN-γ to augment the expression of cathepsin B, cathepsin S and MMPs-2 and -14 (FIG. 6). MMP-9 was also induced in am leupeptin-dependent fashion (FIG. 6).

When viewed in combination, these studies demonstrate that cathepsin-dependent apoptosis is a critical stimulator of the inflammatory and the proteolytic responses at sites of IFN-γ-induced emphysema.

The present invention may be embodied in other specific forms without departing from the spirit or essential attributes thereof.

What is clamed is:

1. A method for treating a patient suffering from chronic obstructive pulmonary disease comprising administering to the patient a pharmaceutically effective amount of at least one cathepsin-S inhibitor or cathepsin-B inhibitor, or a combination of at least one cathepsin-S inhibitor and one cathepsin-B inhibitor.

2. The method according to claim 1, further comprising administering an anti-inflammatory agent.

3. A method for preventing a patient from suffering chronic obstructive pulmonary disease comprising administering to the patient a pharmaceutically effective amount of at least one cathepsin-S inhibitor or cathepsin-B inhibitor, or a combination of at least one cathepsin-S inhibitor and one cathepsin-B inhibitor.

4. The method according to claim 3, further comprising administering an anti-inflammatory agent.
